(19) United States
(12) Patent Application Publication
(10) Pub. No.: US 2019/0194686 A1
(43) Pub. Date: Jun. 27, 2019

(54) HOST CELLS WITH ENHANCED PROTEIN EXPRESSION EFFICIENCY AND USES THEREOF

(71) Applicant: Development Center for Biotechnology, Taipei City (TW)

(72) Inventors: HSIN-LIN LU, Taipei City (TW); CHIEN-I LIN, Taipei City (TW); CHAO-YI TENG, Taipei City (TW)

(73) Assignee: Development Center for Biotechnology, Taipei City (TW)

(21) Appl. No.: 16/147,741

(22) Filed: Sep. 29, 2018

Related U.S. Application Data
(60) Provisional application No. 62/610,918, filed on Dec. 27, 2017.

(51) Int. Cl. C12N 15/85; C12N 15/113

(52) U.S. Cl. CPC C12N 15/85; C12N 2015/8518; C12N 15/113

(57) ABSTRACT

A host cell for protein expression having a lower expression level of a gene, as compared to a wild-type cell, wherein the gene is selected from HDAC8, Dab2, Caspase3, Sys1, Ergic3, Grasp, Trim 23, or a combination thereof. The host cells are CHO cells. The lower expression level of the gene results from RNA interference, which may be achieved by transfecting a vector that contains an shRNA targeting the gene.

### FIELD OF THE INVENTION

The present invention relates to host cells for protein production, particularly to engineered host cells, such as CHO cells, that can produce proteins at higher levels as compared to the wild-type cells.

### BACKGROUND OF THE INVENTION

Protein pharmaceuticals are typically produced by expression in suitable host cells. Chinese Hamster Ovary (CHO) cells are the most widely used host cells for protein drug productions. Optimization of host cells (e.g., by genetic modifications of the host cells) or optimization of downstream processes are being explored to enhance protein drug production efficiencies. Many strategies are currently available for enhancing protein expression and/or secretion, e.g., by using chemical reagents or by genetic modifications of the cells.

With genetic modifications, one typically targets genes that are involved in transcription, post-transcription regulation, translation, and post-translation events. For example, post-transcriptional regulatory elements (PTREs) have been targets for manipulations to enhance protein expression. (see, Mariati et al., “Post-transcriptional regulatory elements for enhancing transient gene expression levels in mammalian cells,” Methods Mol. Biol., 2012, 801: 125-35.)

While prior art techniques for modifying protein-expressing host cells are useful, there is still a need for other methods to enhance protein expression in host cells.

### SUMMARY OF THE INVENTION

Embodiments of the invention relate to new types of host cells that have improved protein (e.g., antibody) production efficiencies. The new types of host cells are genetically engineered to modify one or more genes that were unexpectedly found to impact protein expression or secretion. Such genes are different from previously known genes, such as the post-transcriptional regulatory elements, that have been targets for manipulation to enhance protein expression. Genes manipulated in this invention are not related to transcription, post-transcriptional regulation, translation, or post-translation events. Thus, it was unexpected that suppressing the expression of these genes could lead to enhanced protein expression/secretion.

In accordance with embodiments of the invention, genetic engineering of host cells may involve knockdown of one or more genes selected from HDAC8, Dab2, Caspase3, Sys1, Ergic3, Grasp, and Trim23. Gene knockdown may be accomplished by any suitable genetic engineering techniques known in the art, such as RNA interference with the target genes. RNAi targeting these genes may be performed by transfecting proper constructs into cells to knockdown these target genes to produce the engineered host cells.

In preferred embodiments, the host cells are CHO cells and the target genes may be HDAC8, DAB2, or Caspase3 gene, or a combination thereof. Inhibition or suppression of one or more of these genes with short-hairpin RNA (shRNA) or siRNA produces host cells that can support enhanced protein expression and/or secretion. For example, shRNA inhibition of Caspase3 can lead to reduced apoptosis of the host cells. Therefore, inhibition of the Caspase3 gene by siRNA or shRNA, either by short-term inhibition or long-term inhibition, can increase protein (e.g., antibody) production.

In accordance with some embodiments of the invention, siRNA or shRNA inhibition of HDAC8, DAB2, or Caspase3 may lead to stable cells lines. These stable cell lines may be selected after evaluating their transfection efficiencies, antibody-expression increases, lactate metabolisms, growth rates, adaptability to new media, and/or stabilities over long-term passages (e.g., 60 generations or more). In addition to being able to produce/secret more proteins (e.g., antibodies), these stable cells also have the characteristic of higher stability and being able to adapt to new culture media. Thus, they are suitable for downstream process development.

One aspect of the invention relates to host cells for protein expression. In accordance with one embodiment of the invention, a host cell comprises a lower expression level of HDAC8, Dab2, Caspase3, Sys1, and/or Trim23 gene, as compared to a wild-type cell. For example, the engineered host cells may have a lower expression level manifested as gene knockdown by 15% or more. That is, the knockdown cells may express the particular gene at 85% level or lower, as compared to the wild-type cells.

In accordance with preferred embodiments of the invention, the gene having a lower expression level is HDAC8, Dab2, Caspase3, or a combination thereof. In accordance with some embodiments of the gene, the host cells are CHO cells. In accordance with some embodiments of the invention, the lower expression level of the post-transcriptional regulatory gene or the apoptosis gene results from RNA interference.

Other aspects of the invention will become apparent with the following description, the drawings, and the accompanied claims.

### BRIEF DESCRIPTION OF THE DRAWINGS

FIG. 1 shows protein production levels after knockdown of various genes in 1C9 cells. 1C9 is a low IgG producing cell line (1.28 mg/L at day 6 of Batch culture). The 1C9 cells were used to see if IgG secretion can be boosted by siRNA inhibition of various genes.

FIG. 2 shows candidate genes for knockdown as selected from analysis of high and low production cells using gene arrays from NimbleGene and Agilent.

FIG. 3 shows protein (Avastin and Herceptin) expression levels in various cell lines with knockdown of the target genes.

FIG. 4 shows a non-Lenti viral vector (plasmid) suitable for shRNA constructs.

FIG. 5 shows a plasmid derived from Lenti virus for construction of shRNA.

FIG. 6 shows an example of a sequence format for an shRNA.

FIG. 7 shows selections of cell pools using puromycin at various concentrations.

FIG. 8 shows proteins expression levels in various transfectant cells selected with puromycin, as described in FIG. 7.

FIG. 9 shows a schematic illustrating a procedure for isolating a single clone of the engineered cells.

FIG. 10 shows protein expression levels (transient expression) in the top five single clones having Caspase3 knockdown.

FIG. 11A shows results of analysis of the 5 top single clones with respect to their properties (population doubling time, lactate levels, Caspase3 gene expression levels) in long-term culture (up to 6 weeks).

FIG. 11B shows Caspase3 knockdown levels in the top five single clones at different time during long-term culture.

FIG. 12 illustrates the long-term stabilities of the top 5 single clones, showing the doubling times and viable cell percentages as a function of time (up to 100 generations).

FIG. 13 shows the protein expression levels using three top single clones as a function of time (week 0, week 3, and week 6).

FIG. 14A shows properties of the second-generation cells derived from the top 3 single clones.

FIG. 14B shows the transfection rate of the CHO cells according to embodiments of the invention.

FIG. 14C shows Caspase3 expression levels in the second-generation cells.

FIG. 15 shows protein expression levels in the second-generation cells, as compared with the first-generation cells and the parent cells.

FIG. 16 shows the glycan profiles of Herceptin produced in CHO cells of the invention, indicating that the major glycans include G0F, G1Fa, G1Fb, and G2F, similar to those of the commercial Herceptin.

### DETAILED DESCRIPTION

Embodiments of the invention relate to development of new cell lines for protein productions. These new host cells that have improved protein (e.g., antibody) production and/or secretion efficiencies. Genes manipulated in these cells are not directly related to transcription, post-transcriptional regulation, translation, or post-translation events, and, therefore, it was unexpected that suppressing the expression of these genes could lead to enhanced protein expression/secretion.

By analyzing CHO cell genomes and transcriptomes, inventors of the present invention found that suppression of one or more of HDAC8, Dab2, Caspase3, Sys1, Ergic3, Grasp, and Trim23 genes can lead to CHO cells with improved protein expression and/or secretion. HDAC8 is involved in regulating the structure and organization of chromosomes during cell division. Dab2 is an adapter protein that functions as a clathrin-associated sorting protein (CLASP) required for clathrin mediated endocytosis of selected cargo proteins. Caspase3 is involved in the activation cascade of caspases responsible for apoptosis execution. At the onset of apoptosis, Caspase3 proteolytically cleaves poly(ADP-ribose) polymerase (PARP). Sys1 is a Golgi-localized integral membrane protein homolog and is involved in Golgi transport. Trim23 (tripartite motif-containing 23) plays a role in the formation of intracellular transport vesicles, their movement from one compartment to another. Ergic3 encodes a cycling membrane protein which is an endoplasmic reticulum-Golgi intermediate compartment (ERGIC) protein which interacts with other members of this protein family to increase their turnover. Grasp encodes a protein that functions as a molecular scaffold, linking receptors, including group 1 metabotropic glutamate receptors, to neuronal proteins. None of these genes are directly involved in transcription or translation regulations. Therefore, the fact that suppression of these genes can lead to enhanced protein expression and/or secretion is unexpected.

In accordance with embodiments of the invention, CHO chromosomes and transcriptomes were analyzed to select target genes for engineering. Briefly, gene chips provided by the CHO consortium were analyzed. In one example, a total of four strains of CHO cells without transgenes were analyzed. One strain is the CHO-S production cell line, and the other three strains are suspension CHO cell lines that have been domesticated in the laboratory.

In addition, three pairs of CHO cells with high or low yield characteristics were analyzed. Genes that are expressed at low levels in the high-yield CHO cells, but are expressed at high levels in the low-yield CHO cells are genes that might not be conducive to high level expression of proteins. Therefore, such genes may be candidate targets for RNAi intervention to improve protein expression in the host cells. To investigate whether such genes indeed influence protein expression/secretion efficiencies, the expression levels of these target genes were reduced by RNA interference. Various RNAi constructs for interference with these genes were prepared. These constructs may be transiently transfected into CHO cells to screen for their effects on the protein expression and cell growth and stability.

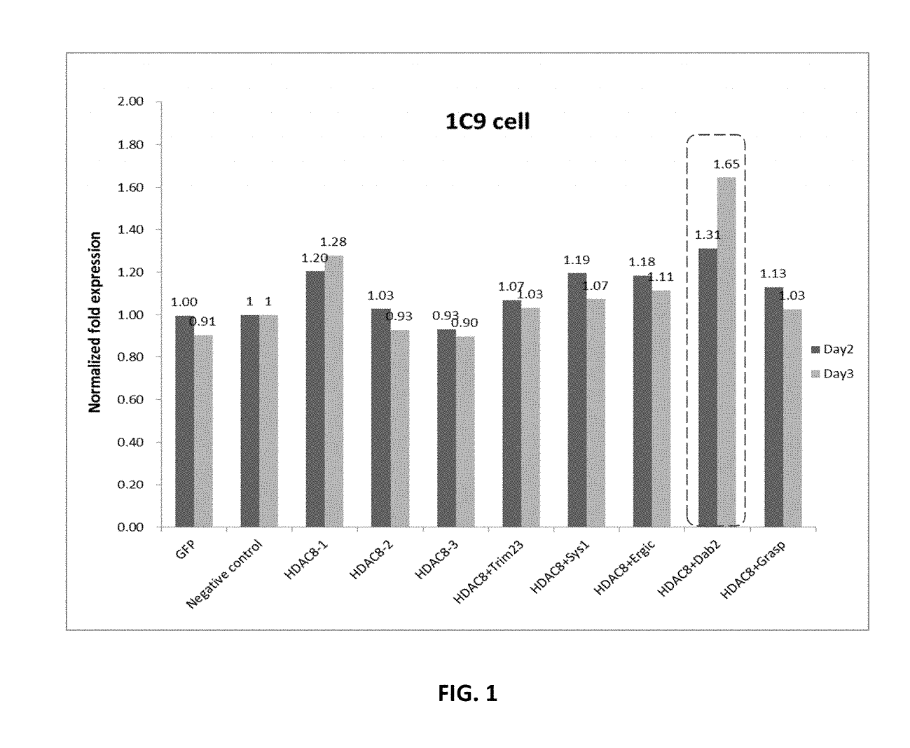

As shown in FIG. 1, knockdowns of HDAC8, Trim23, Sys1, Ergic3, Dab2, and Grasp genes resulted in enhanced protein expression and/or secretion in the modified cells. Furthermore, the combination of HDAC8 and Dab2 knockdown was found to produce the most effects in protein expression improvements. Specifically, knockdown of both HDAC8 and Dab2 genes resulted in cells that can expression proteins from 1.65 folds to 1.8 folds.

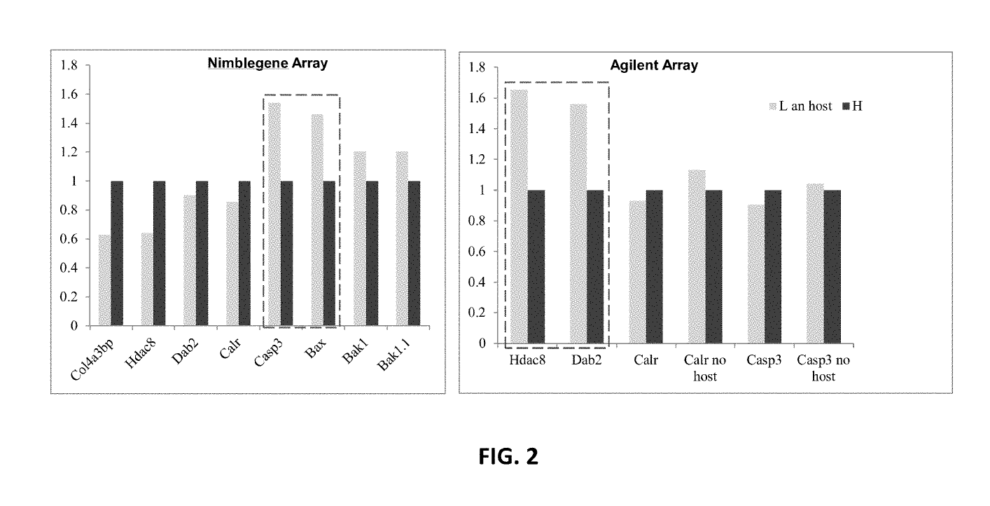

Using a similar approach, two additional CHO cell gene arrays, one from Agilent (Santa Clara, Calif.) and one from Roche NimbleGen (Madison, Wis.), were analyzed. Those genes highly expressed in the low-yield cell lines were identified as targets for interference. From these analyses, two additional genes, BAX and Caspase 3, were identified as knockdown candidates, as shown in FIG. 2.

Based on these target genes, various approaches to RNA interference may be used to suppress their functions. For example, shRNA plasmids containing the target sequences (HDAC8+Dab2, Caspase 3, BAX, and Caspase 3+BAX) may be constructed. Any suitable plasmids or vectors may be used with embodiments of the invention. For example, Lentiviral plasmids or pcDNA3.1(+) vectors are commonly used for shRNA. Commercially available kits may be used, such as the BLOCK-iT™ Lentiviral RNAi Expression System from Thermo Fisher Scientific (Waltham, Mass.).

These plasmids may be amplified in bacteria. Then, the shRNA-containing plasmids were transfected into cell lines of interest (e.g., DXB11-S1). The transfectants may be assessed for transfection efficiency (based on antibiotics resistance in the plasmids, e.g., by determining the killing curves using 0.5-10 μg/ml puromycin) and analyzed for their long-term effects of gene suppression. Gene suppressions of the target genes in these cell lines may be analyzed using real-time PCR. Finally, these cells were analyzed with biochemical, cell or molecular biological assays to investigate the functions of these genes.

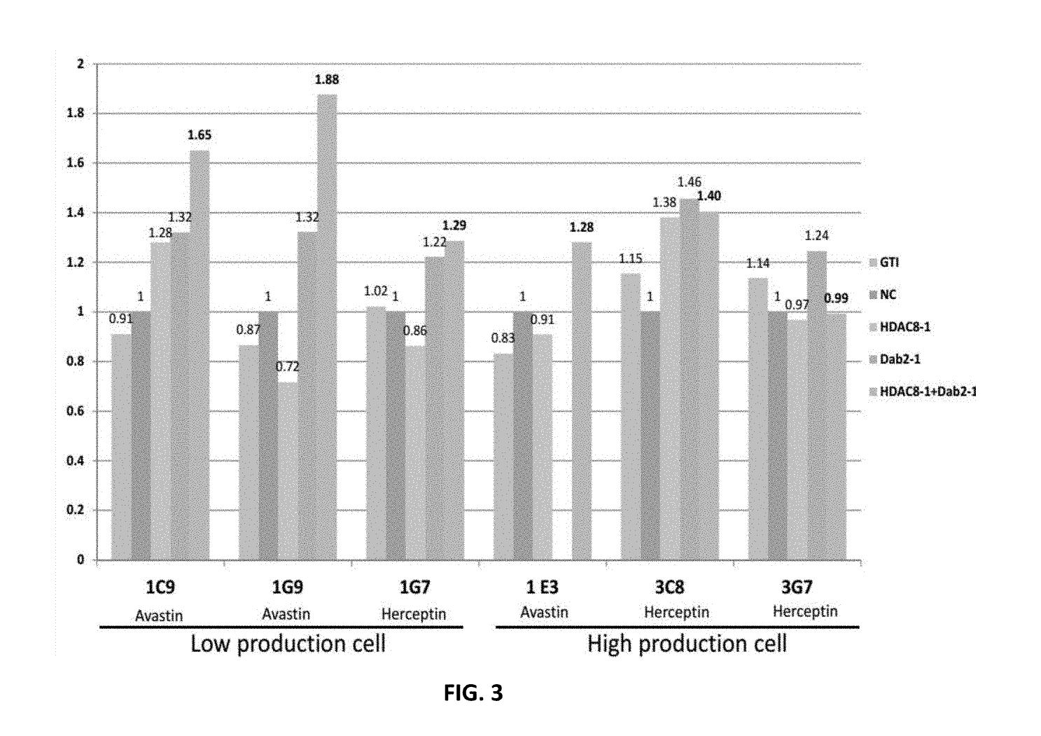

In addition to interference with these genes individually, combination knockdown of these target genes was also investigated. As shown in FIG. 3, a combination of HDAC8 and Dab2 knockdown led to 1.28-fold-1.88-fold increases in the protein expression levels in most cell lines (1C9, 1G9, 1G7, 1E3, and 3C8). The enhancements in the productions are more prominent in the low-production cells (1C9, 1G9, and 1G7), as compared with the high-production cells (1E3, 3C8, and 3G7). These enhancements were seen with different antibodies (Avastin and Herceptin) expressed in these cells, indicating the production enhancement would be applicable to protein productions in general, not limited to specific proteins.

Combination knockdown of two target genes simultaneous may produce synergistic effects. For example, knockdown of HDAC8 and Dab2, respectively, resulted in 1.28-fold and 1.32-fold productions of Avastin in 1C9 cells, whereas a combination knockdown of HDAC8 and Dab2 produced a 1.65-fold expression in the same cells. Similarly, knockdown of HDAC8 and Dab2, respectively, resulted in 0.78-fold and 1.32-fold productions of Avastin in 1G9 cells, whereas a combination knockdown of HDAC8 and Dab2 produced a 1.88-fold expression in the same cells, indicating a synergistic effect.

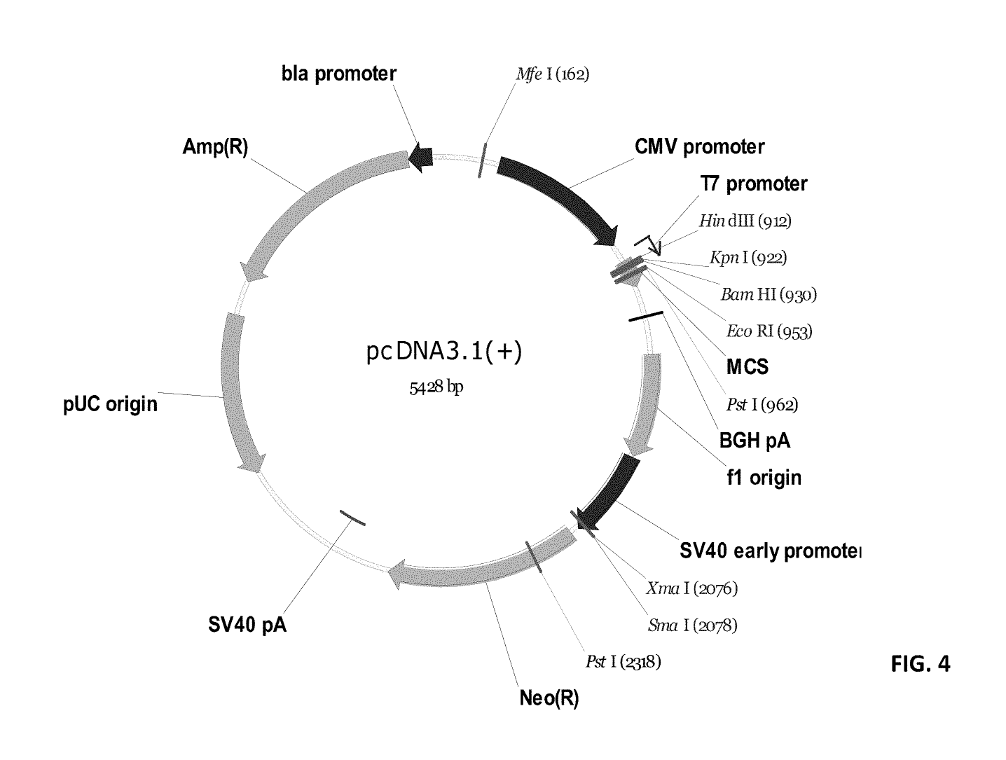

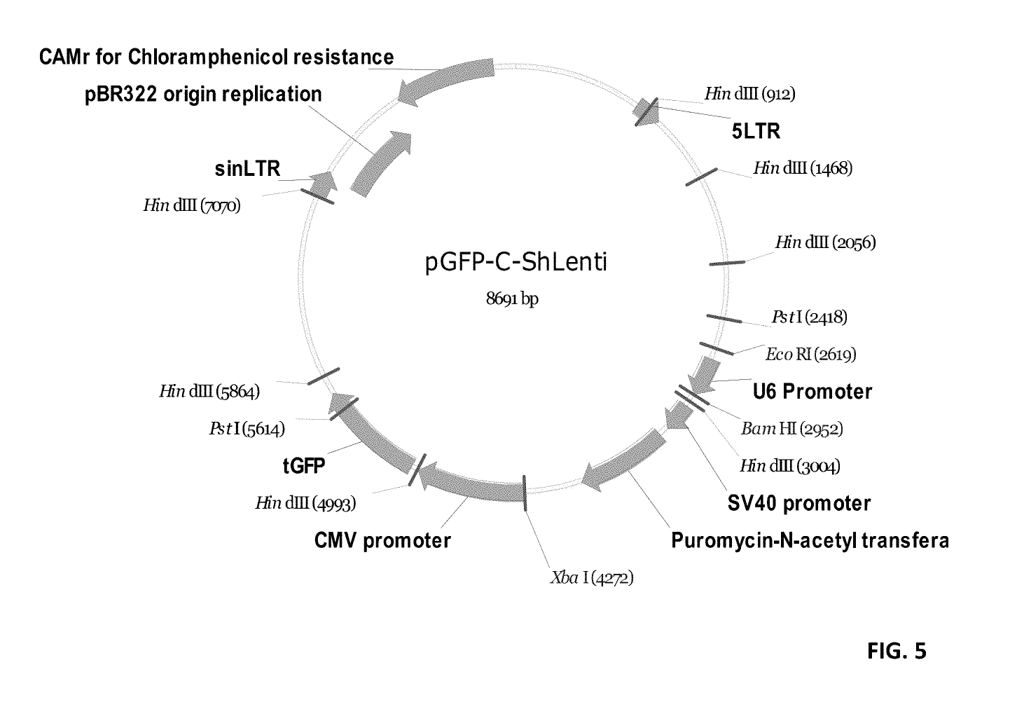

Various shRNA vectors that allow transient and stable transfections, as well as stable delivery of shRNA expression cassettes into host cells, are available. In accordance with embodiments of the invention, transfection of the shRNA constructs into CHO cells may use any suitable vectors, including commercially available vectors such as pcDNA3.1(+) (FIG. 4, available from Thermo Fisher, Waltham, Mass.) and pGFP-C-ShLenti (FIG. 5, available from OriGene, Rockville, Md.). Other suitable vectors known in the art may also be used.

While lentiviral vectors may provide convenient methods to deliver and integrate the snRNA constructs into cell genome, production of pharmaceutical proteins without using Lenti virus elements may be preferable. The following examples demonstrate the use of pcDNA3.1(+) from Thermo Fisher (Waltham, Mass.).

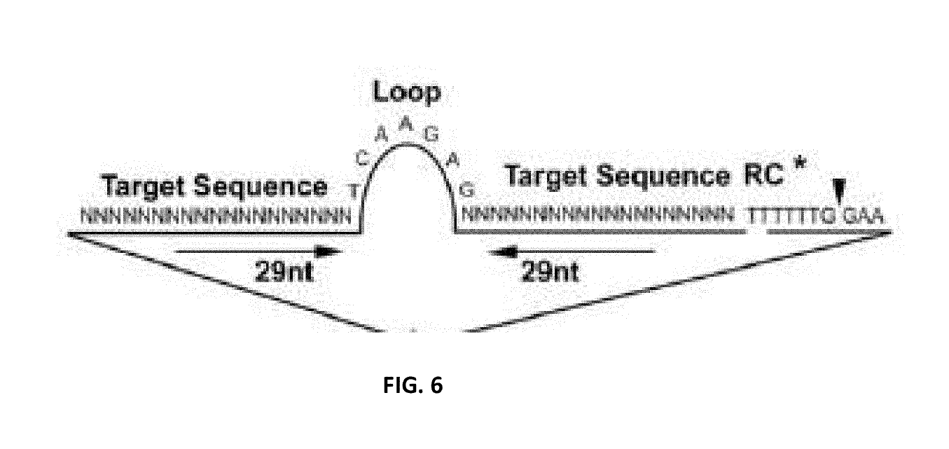

Using pcDNA3.1(+) (FIG. 4) plasmids as examples, a stem-loop sequence/framework having the structure shown in FIG. 6 may be inserted into the plasmids. The oligo in FIG. 6 illustrates a typical stem-loop structure construct. With these plasmids, several constructs have been prepared that contain the sequences for the target genes. The successful constructions of these plasmids may be confirmed with restriction enzyme digests to produce the correct sizes of fragments.

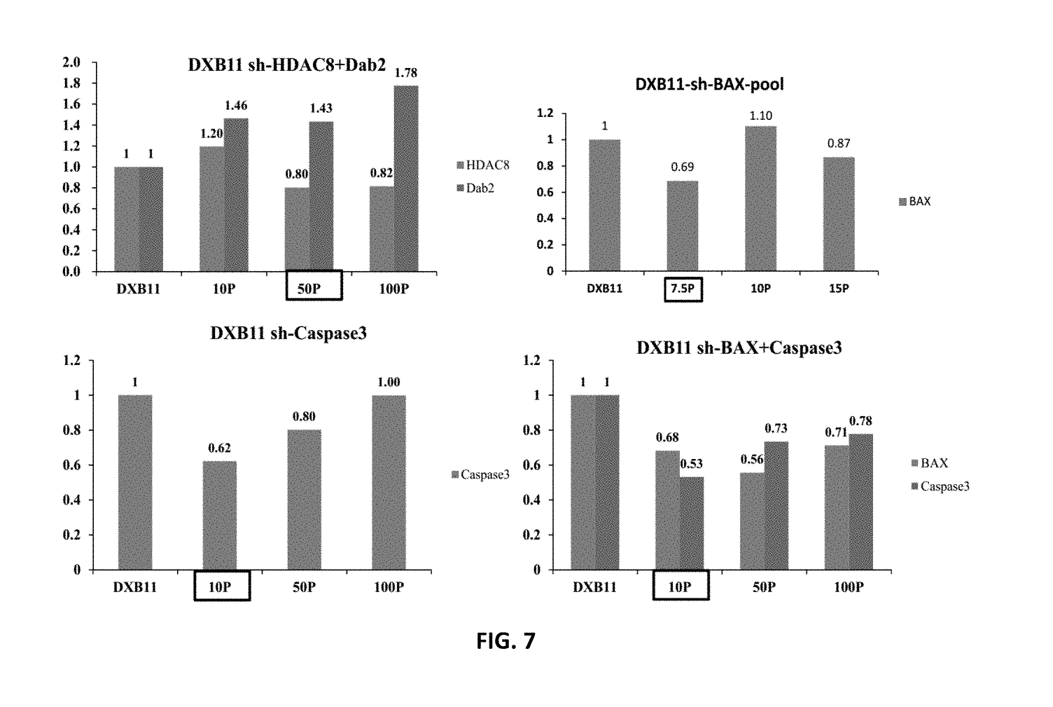

These constructs were used to transfect CHO cells, such as DXB11 cells. The transfected cells were then assessed for their inhibition of the target genes. As shown in FIG. 7, the various transfected cell lines (DXB11 sh-HDAC8+Dab2, DXB11 sh-Caspase3, DXB11 sh-BAX-pool, and DXB11 sh-BAX+Caspase3) may be selected based on selectable markers (e.g., puromycin resistance) and then screened for better inhibitions of the target genes. Briefly, these transfected cell lines were screened with various concentrations of antibiotic (e.g., puromycin) and the suppression of the target genes were assessed with real-time PCR. Based on these screens the best candidate host cells were identified. These best cell lines include, for example, DXB11 sh-HDAC8+Dab2 selected with 50 μg/ml puromycin (HD50P), DXB11 sh-Caspase3 selected with 10 μg/ml puromycin (C10P), DXB11 sh-BAX selected with 7.5 μg/ml puromycin (B7.5P), and DXB11 sh-BAX+Caspase3 selected with 10 μg/ml puromycin (BC10P).

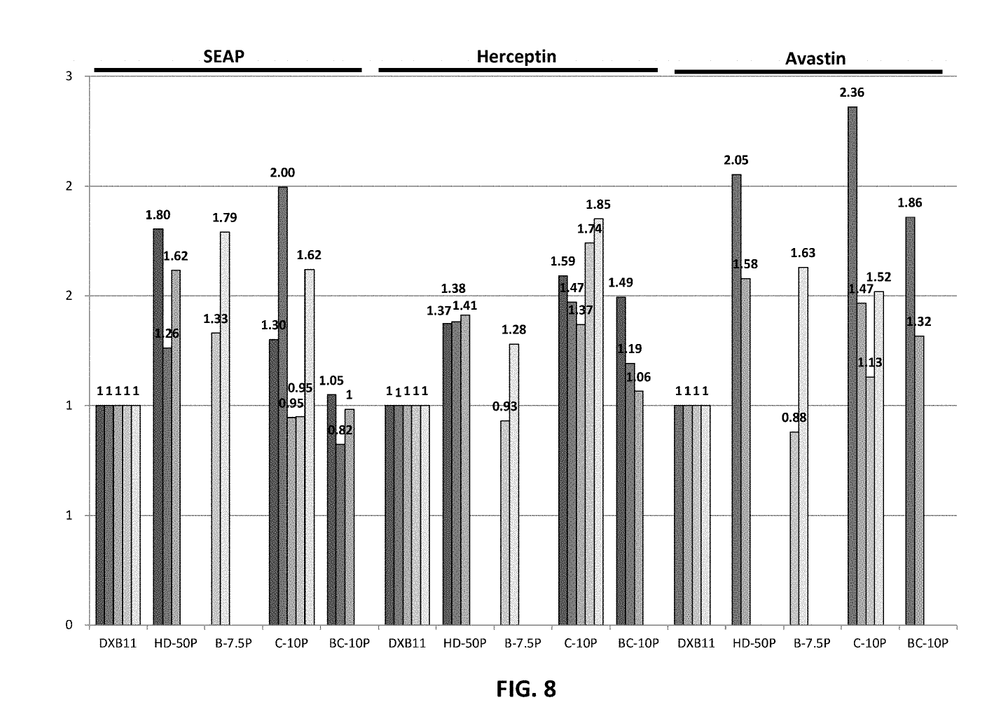

The best candidate host cells were further assessed for their abilities to support enhanced protein productions. These cells were tested with the productions of various proteins, such as SEAP (secreted alkaline phosphatase), Herceptin, and Avastin. As shown in FIG. 8, most of these cell lines did produce more proteins under various culture conditions.

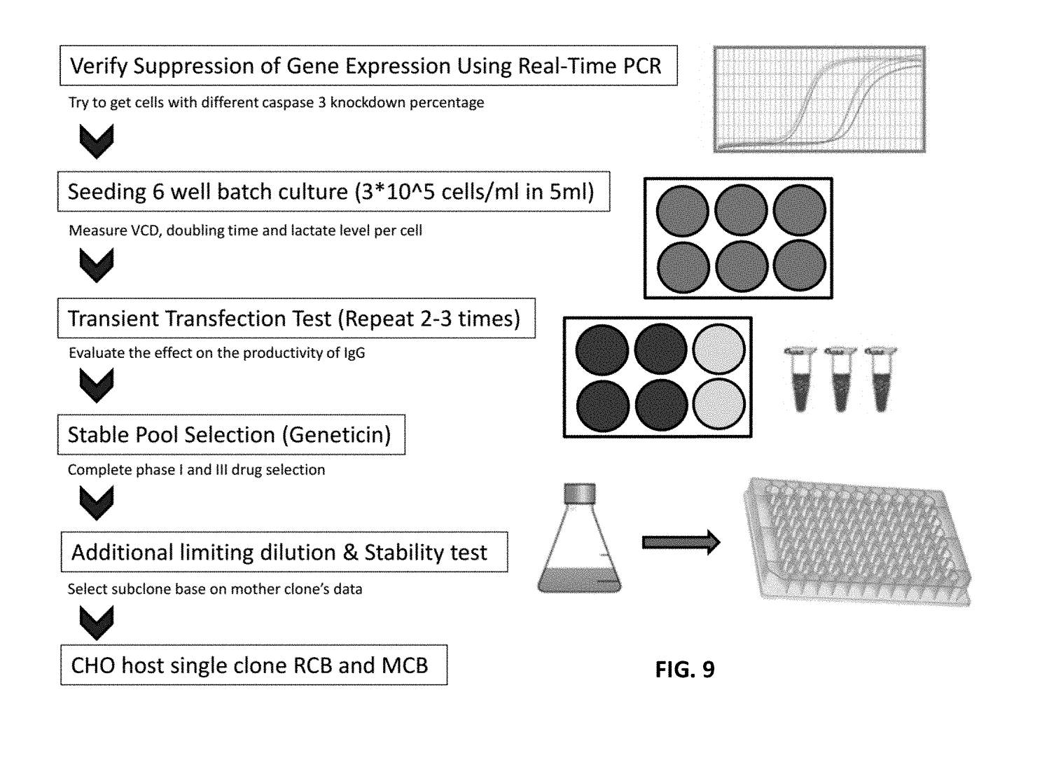

To obtain stable cell lines, these cells may be serially diluted and picked for single clones. Using DXB11 sh-Caspase3 selected with 10 μg/ml puromycin (C10P) cells as an example, single clones are isolated using procedures illustrated in FIG. 9 using ClonePix or any other suitable equipment/protocol. FIG. 9 illustrates one protocol for isolation of single clones. Briefly, after transfection of the cells, the cells are amplified and screened, and then subcloned. Cell lines may be adapted to suspension culture in serum-free, chemically-defined media. Then, a stable pool of transfectants can be generated and characterized prior to the generation of a high-production stable single clone. One skilled in the art would appreciate that this is for illustration only and other procedures for achieving the isolation of single clones may also be used.

As shown in FIG. 9, the suppression of gene expression in these cells may be confirmed using real-time PCR. From these analyses, one can obtain cells with different target gene (e.g., caspase 3) knockdown percentages. Then, these cells are seeded in 6-well plates at a suitable concentration (e.g., 3×10⁵ cells/ml in each 5 ml well) and cultured for an appropriate duration. The viable cell densities (VCD), population doubling times (PDT), and lactate levels of these cells were measured to evaluate the health of the cells.

Next, the effects of target-gene knockdown in these cells on the protein production may be investigated with transient transfection of expression vectors carrying a protein gene (e.g., an IgG). Once these cells are confirmed to support high-level protein expressions, the stable cell pools may be selected by adding a selection drug (e.g., Geneticin or puromycin) to the culture medium. One may first titrate a proper drug concentration to use and then grow the cells at the selected concentration of the drug such that only transfectants with the selected drug resistance marker are viable and can grow at a reasonable rate.

After the stable pool of cells have been selected, these cell pools may be further diluted and their stability tested. For example, these stable cell pools may be subject to limited dilution and select for subclones, the stabilities of which may be assessed.

Finally, if desired, single clones of the stable transfectants may be isolated to establish research cell banks (RCB), from which master cell banks (MCB) or working cell banks (WCB) may be obtained and cryopreserved. Specifically, the single clones may be evaluated based on their properties (e.g., clone stabilities and protein production efficiencies) and the best performing clones will be selected for the creation of RCB. The RCB cells may be further tested and characterized before cryopreserved as MCB.

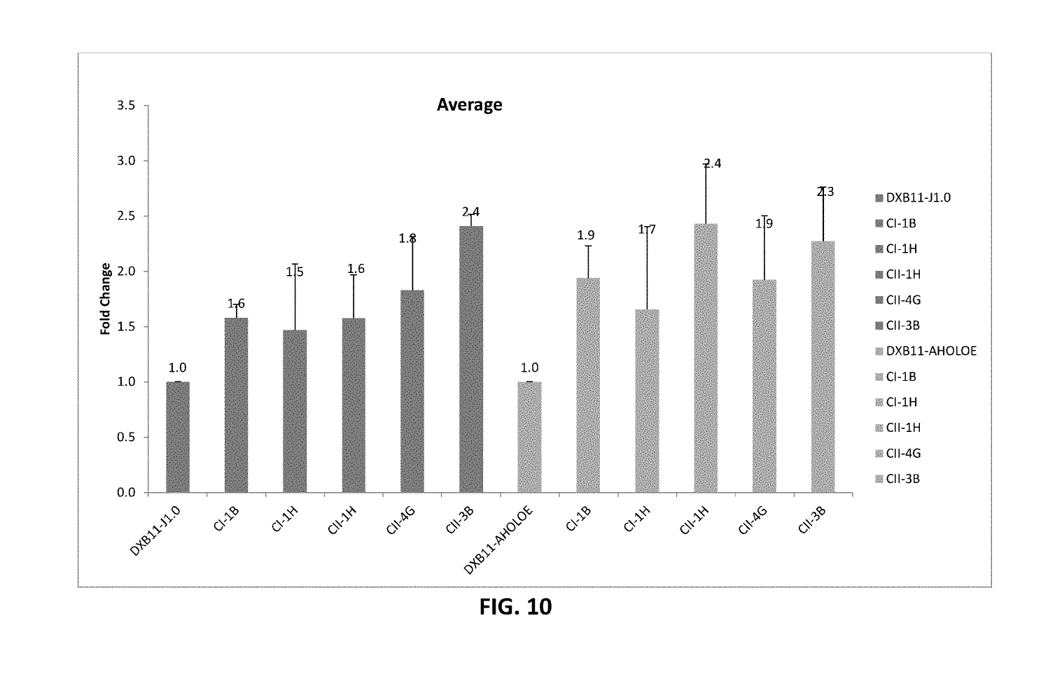

Using top 5 single clones from DXB11-sh-Caspase 3 transfectants as examples, the efficiencies of these cells to support protein expression were investigated. As shown in FIG. 10, all 5 clones (CI-1B, CI-1H, CII-4G, and CII-3B) showed enhanced protein expression after transient transfection of vectors containing Herceptin or Avastin, as compared to the parent DXB11-J1.0 cells. The enhancements range from 1.5 folds to 2.4 folds. These results indicate that suppression of Caspase3 can lead to higher protein production host cells. This finding is unexpected because suppression of caspase-1 was found to result in improved protein folding, but not enhanced protein production or accumulation, in a baculovirus insect cell (sf9 cells) expression system (X. Zhang et al., BMC Biotechnol., 2018 May 2, 18(1):24; doi: 10.1186/s12896-018-0434-1).

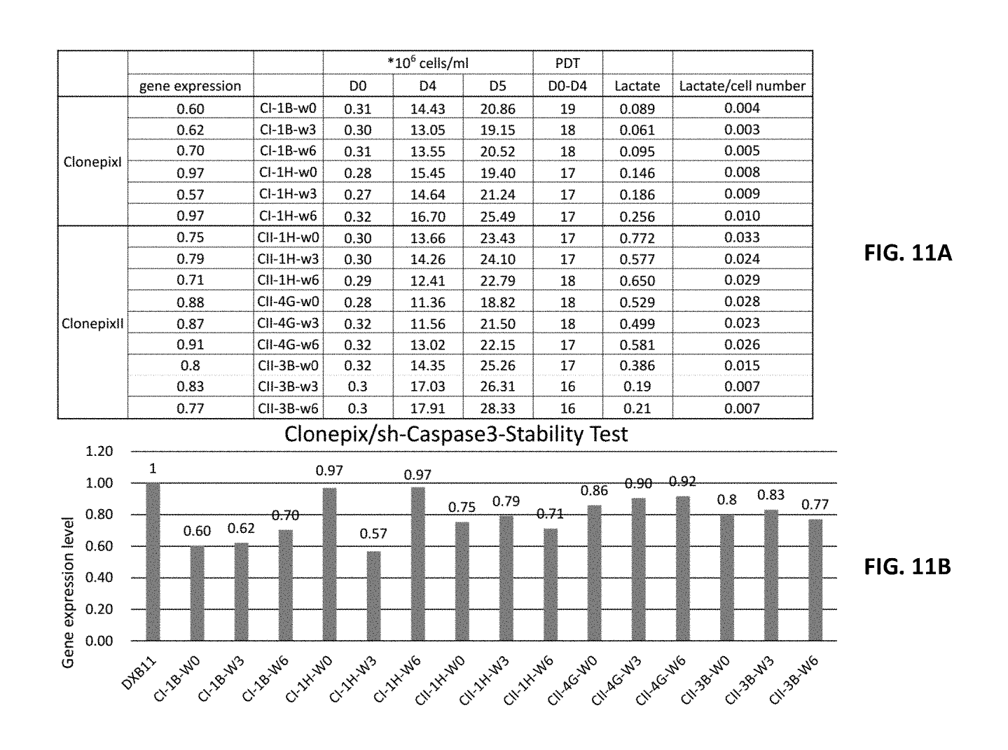

These 5 top clones were further investigated for their long-term behavior. As shown in FIG. 11A, from week 0 to week 6, the cell densities of these 5 clones did not have appreciable changes, regardless of seeding densities (D0, D4, and D5, 0.3-25×10⁶ cells/ml). The long-term stability of cell numbers indicates that these cells have long-term survivability, which is likely due to the suppression of apoptosis.

The population doubling times (PDT) of these cells range from 16-19 hours and did not show appreciable changes over time. These population doubling times are comparable to non-transfected CHO cells under the same conditions. Again, these results indicate that the Caspase3 suppressed cells have substantially the same biological properties as the parent CHO cells.

In fed-batch processes, prolonged culture may result in significant lactate and ammonia accumulation in the culture medium. High levels of lactate are detrimental to cell growth and product quality. CHO cells also show deregulated glucose metabolism associated with high lactate production that can cause medium acidification or undesired osmolality changes. Thus, lactate production may be used as an indicator of cell health.

As shown in FIG. 11A, the lactate levels in these cell cultures did not have significant changes over the 6-week period. The lactate levels, as well as the lactate/cell numbers, were relatively low. The low lactate levels of these top clones suggest that these cells can efficiently utilize the energy source (glucose).

FIG. 11B shows the expression levels of Caspase3 gene. All 5 clones have lower caspase3 expression and the levels did not have substantial changes over the 6-week period. These results indicate that the suppression of caspase3 gene is relatively stable.

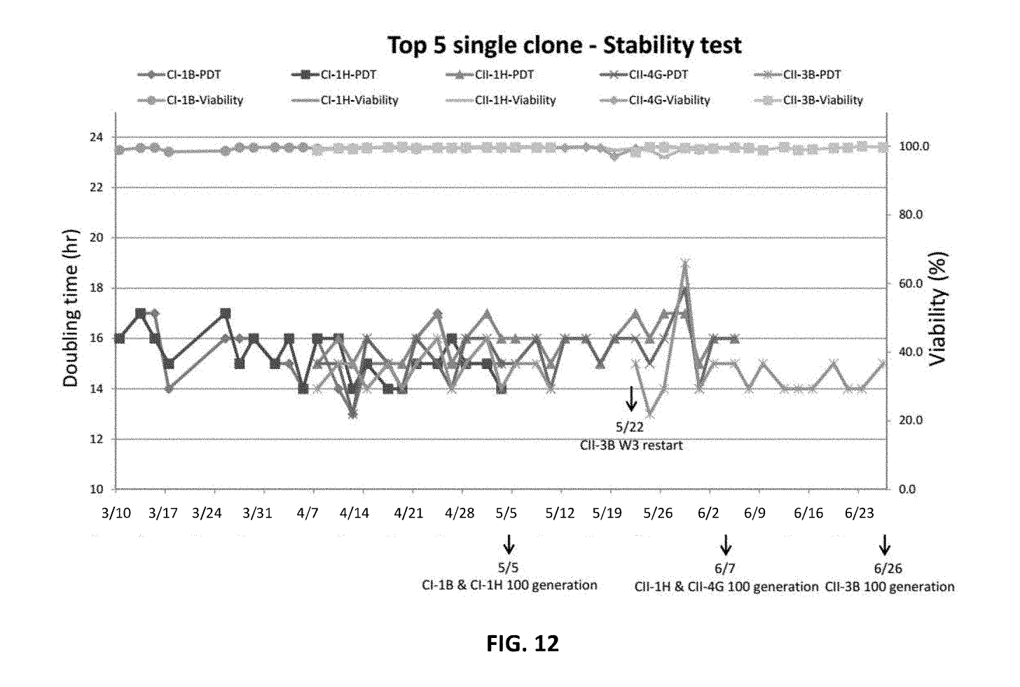

As shown in FIG. 12, these cells maintained near 100% viabilities for a long time (over 100 generations). In addition, the population doubling times (PDT) for these cells were relatively constant. These results all indicate that these transfectant cells are very stable for a long time (e.g., 100 generations or more).

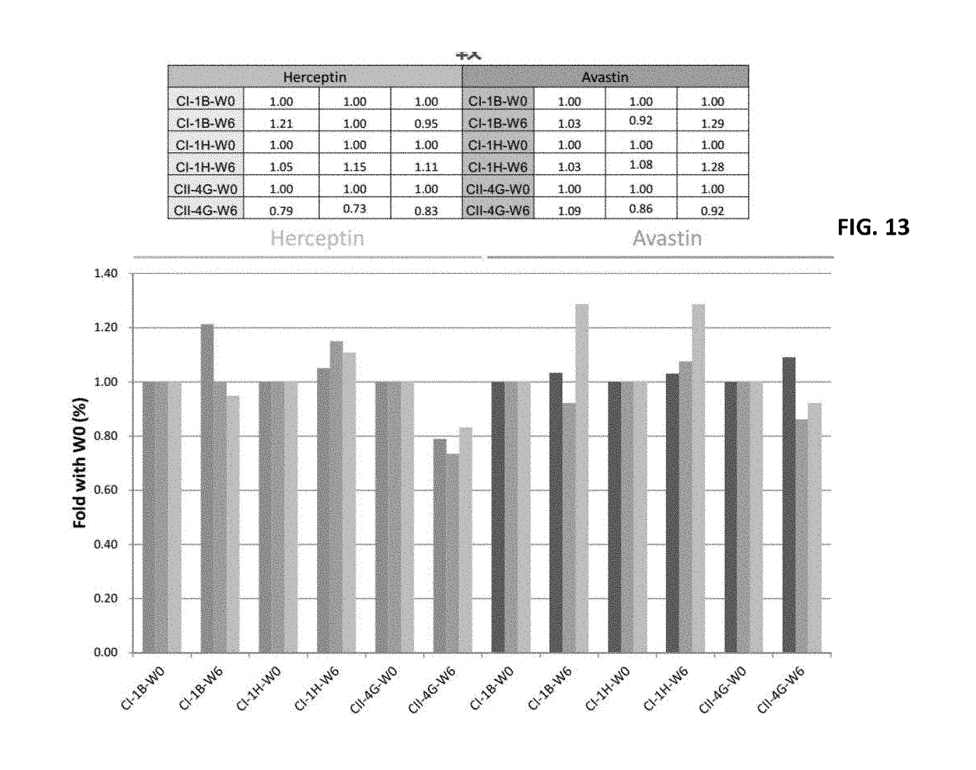

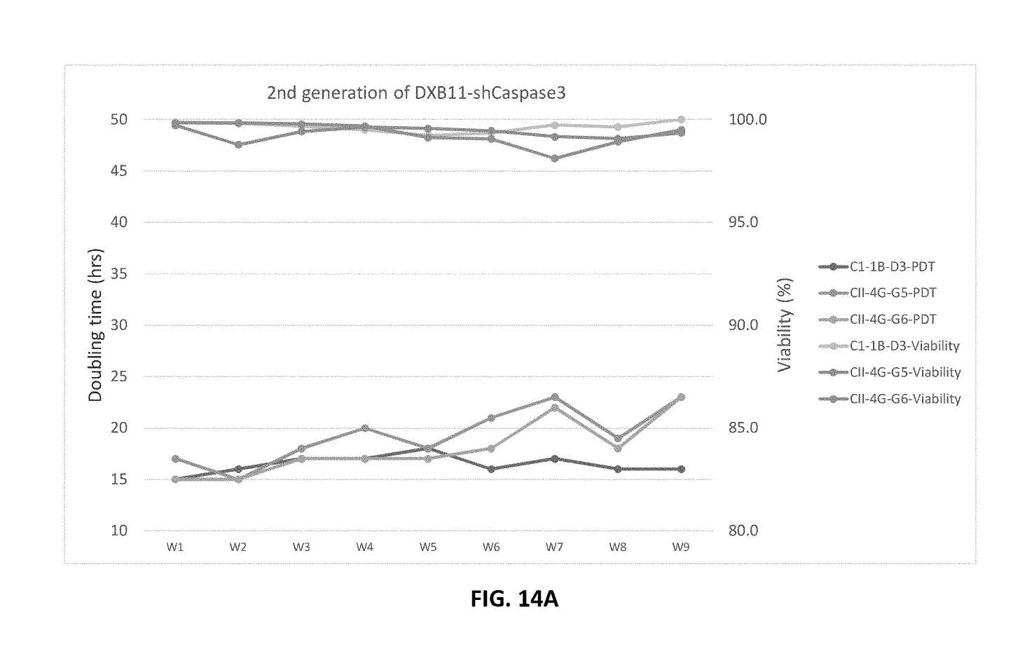

Not only are these cells stable over long terms, but the abilities of these cells to support enhanced protein expression are also very stable. As shown in FIG. 13, the expression levels of both Avastin and Herceptin were maintained at substantially the same levels, except for the CII-4G clone, which showed some reduction in the expression levels at week 6.

To further investigate the long-term health of these cells, second generation of these cells were generated by serial dilution and picking single clones as described above. These second-generation cells were also evaluated for various properties.

TABLE 1

| gene | | *10⁶ cells/ml | | | PDT | | Lactate/ |
|---|---|---|---|---|---|---|---|
| expression | | D0 | D4 | D5 | D0-D4 | Lactate | cell |
| 0.83 | CI-1B-D3 | 0.26 | 8.20 | 18.13 | 19 | 0.777 | 0.043 |
| 0.58 | CI-1B-E2 | 0.29 | 7.54 | 16.63 | 20 | 0.81 | 0.049 |
| 1.05 | CI-1B-F6 | 0.34 | 7.59 | 17.19 | 21 | 1.072 | 0.062 |
| 0.89 | CI-1B-G5 | 0.34 | 5.68 | 13.00 | 23 | 0.791 | 0.061 |
| 1.33 | CII-4G-F6 | 0.29 | 6.43 | 14.29 | 21 | 0.355 | 0.025 |
| 0.31 | CII-4G- | 0.32 | 9.69 | 21.25 | 19 | 0.066 | 0.003 |
| | G6 | | | | | | |

TABLE 1 shows results from analyses of 4 second-generation cells (CI-1B-D3, CI-1B-E2, CI-1B-F6, and CI-1B-G5) derived from the first-generation cell CI-1B, and 2 second-generation cells (CII-4G-F6 and CII-4G-G6) derived from the first-generation cell CII-4G. The population doubling times (PDT) for these second-generation cells are similar to those of the first-generation cells, indicating slightly lower growth rates for the second-generation cells. However, the difference is very small. In addition, the lactate levels, as well as lactate levels per cell, were also slightly higher in the second-generation cells.

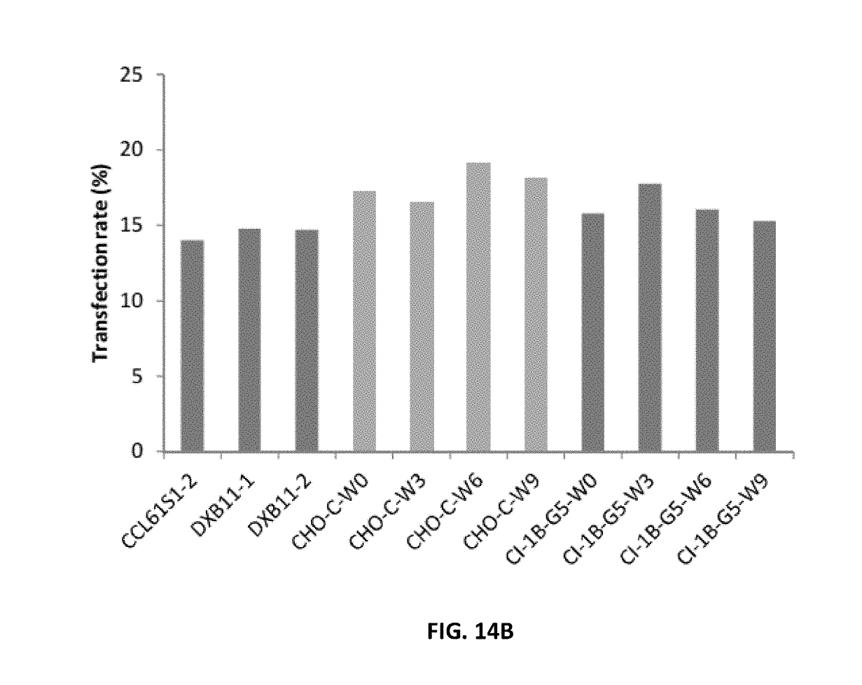

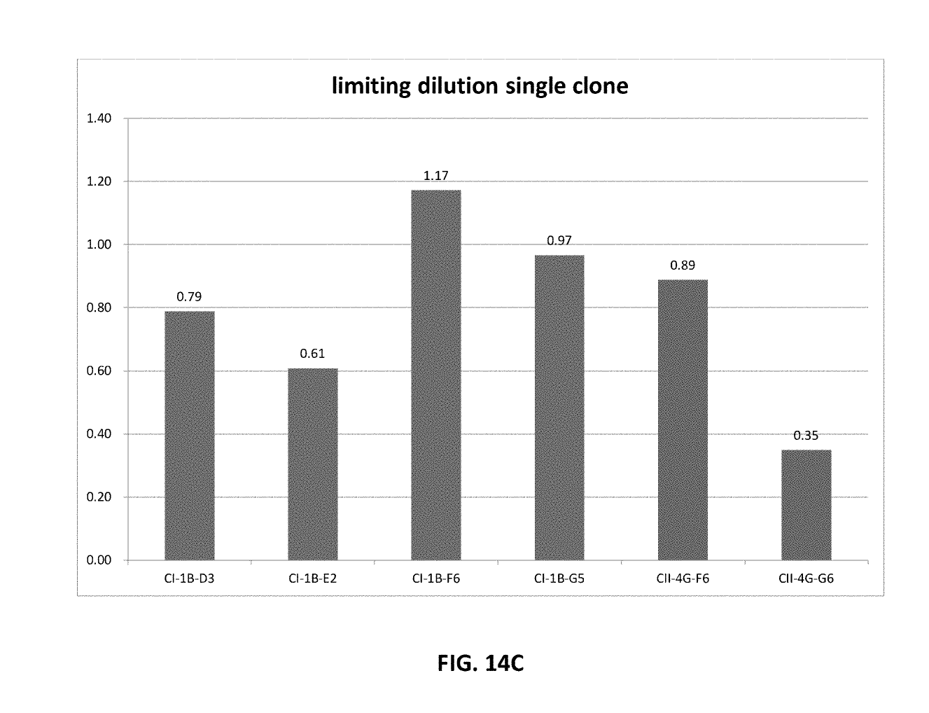

FIG. 14A shows the properties of three exemplary second-generation clones, C1-1B-D3, CII-4G-G5, and CII-4G-G6. The long-term stabilities of these second-generation clones are evidenced by almost 100% viabilities after 9 weeks of cultures. In addition, these cells maintain consistent transfection efficiencies over time. As shown in FIG. 14B, CHO-C (i.e., CII-4G-G6) and CI-1B-G5 cells maintained similar transfection rates (about 15%) from 0 to 9 weeks.

The caspase3 gene expression levels in these second-generation cells, as assessed with real-time PCR, vary more than the first-generation cells (FIG. 14C). For example, CI-1B-D3 and CI-1B-E2 cells still show very good suppression of caspase 3 gene, while CI-1B-F6 and CII-4G-F6 have little changes from the first-generation. Interestingly, CII-4G-G6 cells has a significantly improved suppression of the caspase 3 gene, whereas CI-1B-F6 cells lost the ability to suppress caspase 3 expression.

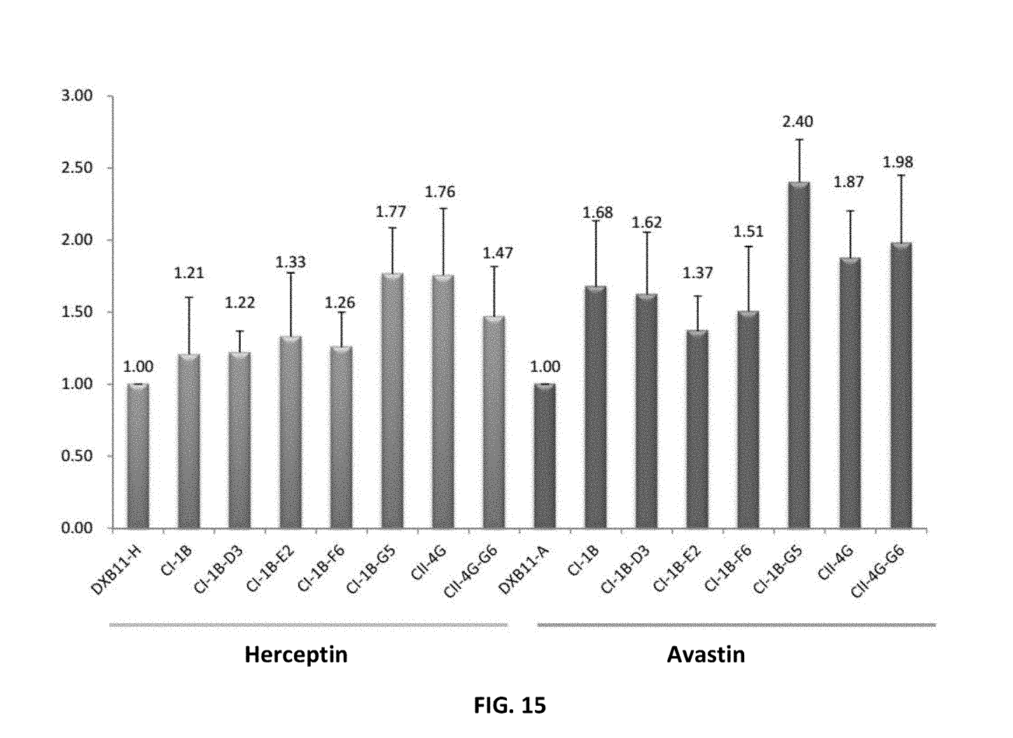

FIG. 15 shows results from evaluation of the second-generation cells to support enhanced expression of antibodies (Avastin and Herceptin). As shown, these second-generation cells show 1.21-2.4 folds of expression levels of these antibodies, as compared to that of the control cells (DXB-11).

The above results clearly show that knockdown of Caspase3, as well as HDAC8, Dab2, Sys1, and Trim23, can produce CHO cells with enhanced production abilities. These enhanced abilities have been found with various CHO cell lines, including CHO-DXB11, CHO-S, CHO-K1, and CHOC cells. In addition, various proteins have been expressed in these cells, including antibodies against Her2, mesothelin (MSLN), and T-cell immunoglobulin and mucin-domain containing-3 (Tim3). In general, these cells can produce proteins (antibodies) at level of about 200 mg/L or higher.

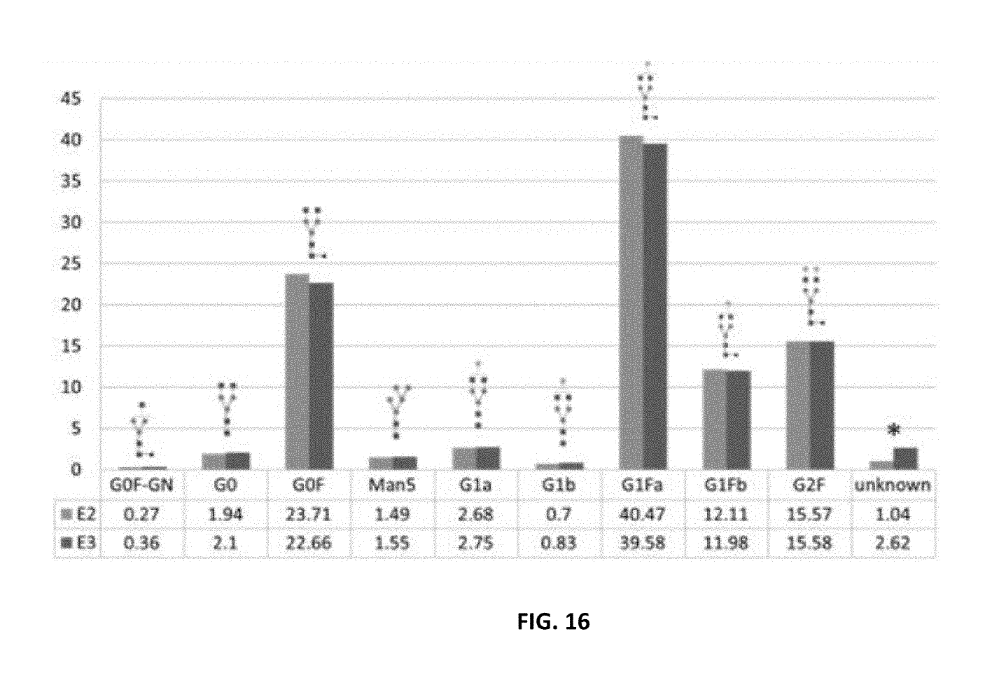

Proteins expressed in these cells have normal properties, including post-translational modifications. As an example, Herceptin expressed in DXB11 and CHOC cells were compared with commercially available Herceptin and were found to found to have similar molecular weights (about 145 KDa). RP-HPLC (reduced and non-reduced) revealed similar banding patterns of the Herceptin produced with these cells, as compared with those of the commercial Herceptin/Trastuzumab. FIG. 16 shows N-linked glycan profiles (after PNGase F release) of Herceptin produced in CHOC cells, as analyzed with an ACQUITY UPLC BEH Glycan column (1.7 μm; Waters Corp., Milford, Mass., USA) and eluted with a gradient of acetonitrile and 50 mM ammonium formate. The glycan analysis revealed that this protein contains mainly G0F, G1Fa, G1Fb, and G2F glycans.

The above description clearly demonstrates the embodiments of the invention. Genes that have no direct connection with protein production were unexpectedly found to impact protein expression and/or secretion. In accordance with embodiments of the invention, these gene are selected as targets for RNAi. RNAi targeting these genes are performed by transfecting proper constructs into cells to knock-down these target genes to engineer the host cells (e.g., CHO cells).

Embodiments of the invention show that inhibition of selected target genes (e.g., HDAC8, Dab2, and Caspase3 genes) by siRNA and shRNA, either by short-term inhibition or long-term inhibition, can produce cells that can support increased protein expression and/or secretion. These results indicate that the selected gene-inhibited host cells have great potential as new hosts for protein drug productions. While specific examples use Caspase3 to illustrate the embodiments of the invention, other genes (particularly, HDAC8, Dab2, and HDAC8+Dab2) may also be targets for knockdown to improve protein productions and/or secretion.

Methods for various procedures are known in the art. The following description provides exemplary procedures and various examples to illustrate embodiments of the invention. One skilled in the art would appreciate that these specific examples are for illustration only and that other variations and modifications are possible without departing from the scope of the invention. For example, the following uses HDAC8, Dab2, BAX, and Caspase3 as examples to illustrate embodiments of the invention. However, other genes may be used without departing from the scope of the invention.

### Cell Culture and Media

Chinese Hamster Ovary (CHO) cell lines DXB11 were obtained from Dr. Lawrence Chasin, University of Columbia. Cell cultures were carried out in an incubator under 5% CO₂, at a temperature of 37° C. and 95% humidity. The media for the cell cultures include Hyclone and Mixed medium (50% CDFortiCHO and 50% ActiCHO). Cell counts and viability analysis were performed after staining with trypan blue using an automatic cell counter TC10 (Bio-Rad, USA).

The transfection constructs include puromycin resistance gene. Stable pools were selected based on puromycin resistance. Once it becomes a single clone, selection is not necessary.

### Vector Constructions

In this example, HDAC8, Dab2, BAX, and Caspasae3 were elected as target genes for RNA interferences. The particular sequence fragments from these genes selected for RNA interference are shown in Table 2:

TABLE 2

| Target | Target Sequence | SEQ ID NO |
|---|---|---|
| HDAC | 5′-GCATACAGGATGAGAAGTA-3′ | 1 |
| Dab2 | 5′-CAGCAAAGCAGAAGAGAAT-3′ | 2 |
| BAX | 5′-CCAAAGTGCCCGAGCTAAT-3′ | 3 |
| Caspase3 | 5′-CGATAGAATTTGAGTCCTT-3′ | 4 |

These target sequences were cloned into pcDNA3.1(+) vectors (FIG. 5) to generate shRNA plasmids for use to suppress these genes to generate the engineered host cells.

The plasmid constructions are as follows: Design separate primers to contain Mfei restriction site at the 5″ end and KpnI restriction site at the 3′ end. Using pGFP-C-Dab2 and pGFP-C-BAX as the templates, perform PCR to amplify the desired fragments (corresponding to the desired RNA sequences) containing a U6 promoter and a puromycin selection gene. GFP-C-Dab2 and p-GFP-C-BAX are HuSH shRNA plasmids constructed by OriGene by cloning the polynucleotides corresponding to the desired RNA sequences into pGFP-C-shLenti vectors (FIG. 6).

Similar primers with EcoRI restriction site at the 5′ end and XmaI restriction site at the 3′ end were separately designed. In a similar manner, PCR were performed using pGFP-HDAC8 and pGFP-C-Caspase 3 as templates to amplify the desired fragments (corresponding to the desired RNA sequences) containing a U6 promoter and a puromycin selection gene. Table 3 shows various primer sequences:

TABLE 3
Oligo Sequences

| | | | SEQ ID |
|---|---|---|---|
| # | Name | Sequence | NO |
| 1 | shLenti-U6-Forw-MfeI-I | 5′AACAATTgCCCAgTggAAAgACgCgC3′ | 5 |
| 2 | shLenti-U6-Forw-EcoRI-II | 5′AAgAATTCCCCAgTggAAAgACgCgC3′ | 6 |
| 3 | shLenti-Rev-Puro-kpnI-I | 5′AAggTACCAACTgCTgAgggCTggAC3′ | 7 |
| 4 | shLenti-Rev-Puro-XmaI-II | 5′ATACCCgggAACTgCTgAgggCTggAC3′ | 8 |

The PCR fragments were temporarily cloned into pJET1.2 vectors (Thermo Fisher), and the sequences were confirmed. Use restriction enzymes MfeI/KpnI and EcoRI/XmaI, respectively, to cut the HDAC8, Dab2, BAX, and Casepase3 fragments from the temporary pJET1.2 vectors.

Cut pcDNA3.1(+) vector with restriction enzymes EcoRI and XmaI. Then, clone HDAC8 and Caspase3 fragments, respectively, into the vectors to obtain pcDNA3.1(+)-HDAC8 and pcDNA3.1(+)-Caspase3 vectors.

Cut pcDNA3.1(+)-HDAC8 vector with restriction enzymes MfeI and KpnI, and then construct the Dab2 fragment into the cut vector to obtain pcDNA3.1(+)-Dab2-HDAC8 vector.

In addition, pcDNA3.1(+) and pcDNA3.1(+)-Caspase3 vectors were cut with restriction enzymes MfeI and KpnI. Then, the BAX fragment was ligated into the cut vector to obtain pcDNA3.1(+)-BAX and pcDNA3.1(+)-BAX-Caspase3. After construction of these vectors, the proper constructions were confirmed by restriction enzyme digestions to confirm that the proper fragments (i.e., proper sizes) were constructed into the vectors.

### Transfection of CHO Cells

DXB11 cells were cultured in a 6-well plate. Each well has 3×10⁶ cells in 3 mL of Hyclone™ HyCell™ CHO culture medium (GE Healthcare) containing 8 mM GlutaMAX™ (Thermo Fischer).

Transfections of the vectors (e.g., pcDNA3.1) containing the shRNA constructs may be performed using any suitable reagents known in the art, such as a lipophilic agent FreeStyle MAX (Thermo Fischer). For example, the RNAi/shRNA vectors and the transfection reagent FreestyleMAX™ (Thermo Fischer) were separately added into OptiPRO™ SFM (Thermo Fischer) to prepare the vector solutions as shown in TABLE 4. These solutions were let stand for 5 minutes before they were added into the transfection reagents and mixed well. The resultant solutions were allowed to stand for 20 minutes before transfection into the cells. The cells were then evaluated 3 days after the transfection.

TABLE 4
RNAi Vector Transfections

| | Stock | RNAi | | Freestyle | | |
|---|---|---|---|---|---|---|
| RNAi | Conc | (50 nM) | OPTI | MAX | OPTI | Cell |
| siGLO Green Transfection | 5 nM | 10.0 | 140.0 | 3 | 147 | 1C9 |
| Indicator | | | | | | |
| Non-targeting siRNA | 5 nM | 10.0 | 140.0 | 3 | 147 | 1E3 |
| siRNA | 5 nM | 10.0 | 140.0 | 3 | 147 | 2 × 10⁶ 2 ml |

shRNA Vector Transfections

| shRNA | Conc. | DNA | DNA | | | Freestyle | | |
|---|---|---|---|---|---|---|---|---|
| plasmid | ng/μg | 16.6 ug | volume | OPTI | tube | MAX | OPTI | cell |
| pcDNA3.1- | 3659.9 | 4.54 | 100.0 | 400.0 | 500.0 | 16.6 | 483.4 | DXB11 |
| Dab2-HDAC8 | | | | | | | | |
| pcDNA3.1- | 3249.2 | 5.11 | 100.0 | 400.0 | 500.0 | 16.6 | 483.4 | |
| BAX | | | | | | | | |
| pcDNA3.1- | 2850.3 | 5.82 | 100.0 | 400.0 | 500.0 | 16.6 | 483.4 | 10 × 10⁶ |
| Caspase3 | | | | | | | | 10 m1 |
| pcDNA3.1- | 2892.7 | 5.74 | 100.0 | 400.0 | 500.0 | 16.6 | 483.4 | |
| BAX- | | | | | | | | |
| Caspase3 | | | | | | | | |

### Protein Expression

For protein/antibody production in the test CHO cells, the antibody/protein expression constructs may be from commercial sources or be prepared based on procedures known in the art and transfected into the test CHO cells for transient expression of the antibodies or proteins. The transfected CHO cells were cultured for an appropriate duration (e.g., 3 days) to produce the antibodies.

The protein expression levels may be assessed using any suitable methods. For example, a GreatEscAPe™ chemiluminescence kit was obtained from Clontech. Prepare 1× Dilution Buffer by diluting the 5× Dilution Buffer 1:5 with ddH₂O.

To evaluate the protein expression levels, transfer 25 μl of cell culture medium from transfected cells or mock transfected cells to a 96-well microtiter plate. If necessary, the plate can be sealed and frozen at −20° C. for future analysis. Add 75 μl of 1× Dilution Buffer to each sample in the 96-well microtiter plate. Seal the plate with adhesive aluminum foil or a regular 96-well lid and incubate the diluted samples for 30 min at 65° C. using a heat block or water bath.

Cool the samples on ice for 2-3 min, then equilibrate to room temperature. Add 100 μl of SEAP Substrate Solution to each sample. Incubate for 30 min at room temperature before reading. Use a 96-well plate reader luminometer (e.g., CLARIOstar®) to detect and record the chemiluminescence signals.

### Knockdown Analysis Using Real-Time PCR

The target gene expression levels may be evaluated with QPCR. Briefly, RNA from the cells was extracted using RNA purification reagents (from Qiagen) and quantified using NanoDrop 2000. The QPCR reactions were performed using the following conditions (TABLE 5):

TABLE 5

| | | |
|---|---|---|
| Power SYBR ® Green RT-PCR Mix (2X) | 5 | μl |
| 10 μM of forward primer (TABLE 4) | 0.5 | μl |
| 10 μM of reverse primer (TABLE 4) | 0.5 | μl |
| RT Enzyme Mix (125X) | 0.08 | μl |
| Template gDNA | 50 | ng |
| UltraPure water | To 10 | μl |

Using StepOne real-time PCR machine, perform QPCR with the following protocols (TABLE 6):

TABLE 6

| Stage | Step | Temp | Time |
|---|---|---|---|
| Holding | Reverse transcription | 48° C. | 30 min |
| Holding | Activation of AmpliTaq Gold ®DNA | 95° C. | 10 min |
| | Polymerase, UP (Ultra Pure) | | |
| Cycling | Denature | 95° C. | 15 sec |
| (40 cycles) | Anneal/Extend | 60° C. | 1 min |
| Melt curve | Denature | 95° C. | 15 sec |
| (Optional) | Anneal | 60° C. | 15 sec |
| | Denature | 95° C. | 15 sec |

To evaluate the expression levels of apoptotic genes (Caspase3 and BAX) and other genes (e.g., HDAC8 and DAB2), the following primers were used (TABLE 7):

TABLE 7

| Gene | Primers | Length | Primer′s sequence (5′→3′) | SEQ ID No |
|---|---|---|---|---|
| HDAC8 | HDAC8-F | 24 | CTATGCAGCAGCTATAGCAGGAGC | 9 |
| | HDAC8-R | 22 | CTTTCTTTGCGTGATGCCACCC | 10 |
| | HDAC8-F-2 | 25 | CCAGGAACAGGTGACATGTCTGATG | 11 |
| | HDAC8-R-2 | 23 | GGTGTTGGAGCCTTTCACAGATG | 12 |
| Dab2 | Dab2-F | 23 | GGCCAGATTCAAAGGTGATGGTG | 13 |
| | Dab2-R | 27 | CATCATAGAATCTTGGCTCATTTTGTC | 14 |
| | Dab2-F-2 | 26 | GTGCAGCTTAAACCTAAACATTCTAC | 15 |
| | Dab2-R-2 | 24 | GAGATGGTCCCATGTGCTTTTGAG | 16 |
| BAX | Bax-F | 24 | ATGAGCTGGACAGCAACATGGAGC | 17 |
| | Bax-R | 25 | AGTTGAAGTTGCCATCAGCAAACAT | 18 |
| | Bax-F-2 | 19 | CCGAGAGCGGCTGCTTGTC | 19 |
| | Bax-R-2 | 25 | GCCACAAAGATAGTCACTGTCTGCC | 20 |
| Caspase3 | Caspase3-F | 26 | GGAGAACAATGAAACCTCAGTAGATT | 21 |
| | Caspase3-R | 27 | CTATTGTCCAGATACATTCCAGAGTCC | 22 |
| | Caspase3-F-2 | 24 | CACCAGAGATTAAAGGACCATGGC | 23 |
| | Caspase3-R-2 | 21 | GGAAGCCTGGAGCACCACCTC | 24 |
| | Caspase3-F-3 | 20 | CAAAGGACGGGTCCTGGTTC | 25 |
| | Caspase3-R-3 | 22 | CCTTCCGGTTAACTCGAGTAAG | 26 |
| | Caspase3-F-4 | 22 | CTTACTCGAGTTAACCGGAAGG | 27 |
| | Caspase3-R-4 | 23 | GCATGGACACAATACACGGAATC | 28 |

While embodiments of the invention have been illustrated with limited number of examples, one skilled in the art would appreciate that other modifications and variations are possible without departing from the scope of the invention. Therefore, the scope of protection of this invention should only be limited by the attached claims.

What is claimed is:

1. A host cell for protein expression, wherein the host cell comprises a lower expression level of a gene as compared to a wild-type cell, wherein the gene is selected from the group consisting of HDAC8, Dab2, Caspase3, Sys1, Ergic3, Grasp, Trim 23, and a combination thereof.

2. The host cell according to claim 1, wherein the gene is selected from the group consisting of HDAC8, Dab2, Caspase3, and a combination thereof.

3. The host cell according to claim 1, wherein the gene is Caspase3.

4. The host cell according to claim 1, wherein the host cell is a Chinese Hamster Ovary (CHO) cell.

5. The host cell according to claim 1, wherein the lower expression level of the gene results from RNA interference.

6. The host cell according to claim 5, wherein the RNA interference is achieved by transfection using a vector comprising an shRNA targeting a gene selected from the group consisting of HDAC8, Dab2, Caspase3, and a combination thereof.

7. The host cell according to claim 5, wherein the RNA interference is achieved by transfection using a vector comprising an shRNA targeting Caspase3.

8. A method for producing a protein using the host cell according to claim 1, comprising transfecting an expression vector encoding the protein into the host cell and culturing the transfected host cell.

9. The method according to claim 8, wherein the protein is an antibody.

10. The method according to claim 9, wherein the antibody is an antibody against Her2, an antibody against mesothelin (MSLN), or an antibody against T-cell immunoglobulin and mucin-domain containing-3 (Tim3).


SEQUENCE LISTING (28 sequences)

SEQ ID NO: 1 - DNA, 19 bp, Artificial Sequence
  Synthetic
gcatacaggatgagaagta

SEQ ID NO: 2 - DNA, 19 bp, Artificial Sequence
  Synthetic
cagcaaagcagaagagaat

SEQ ID NO: 3 - DNA, 19 bp, Artificial Sequence
  Synthetic
ccaaagtgcccgagctaat

SEQ ID NO: 4 - DNA, 19 bp, Artificial Sequence
  Synthetic
cgatagaatttgagtcctt

SEQ ID NO: 5 - DNA, 26 bp, Artificial Sequence
  Synthetic
aacaattgcccagtggaaagacgcgc

SEQ ID NO: 6 - DNA, 26 bp, Artificial Sequence
  Synthetic
aagaattccccagtggaaagacgcgc

SEQ ID NO: 7 - DNA, 26 bp, Artificial Sequence
  Synthetic
aaggtaccaactgctgagggctggac

SEQ ID NO: 8 - DNA, 27 bp, Artificial Sequence
  Synthetic
atacccgggaactgctgagggctggac

SEQ ID NO: 9 - DNA, 24 bp, Artificial Sequence
  Synthetic
ctatgcagcagctatagcaggagc

SEQ ID NO: 10 - DNA, 22 bp, Artificial Sequence
  Synthetic
ctttctttgcgtgatgccaccc

SEQ ID NO: 11 - DNA, 25 bp, Artificial Sequence
  Synthetic
ccaggaacaggtgacatgtctgatg

SEQ ID NO: 12 - DNA, 23 bp, Artificial Sequence
  Synthetic
ggtgttggagcctttcacagatg

SEQ ID NO: 13 - DNA, 23 bp, Artificial Sequence
  Synthetic
ggccagattcaaaggtgatggtg

SEQ ID NO: 14 - DNA, 27 bp, Artificial Sequence
  Synthetic
catcatagaatcttggctcattttgtc

SEQ ID NO: 15 - DNA, 26 bp, Artificial Sequence
  Synthetic
gtgcagcttaaacctaaacattctac

SEQ ID NO: 16 - DNA, 24 bp, Artificial Sequence
  Synthetic
gagatggtcccatgtgcttttgag

SEQ ID NO: 17 - DNA, 24 bp, Artificial Sequence
  Synthetic
atgagctggacagcaacatggagc

SEQ ID NO: 18 - DNA, 25 bp, Artificial Sequence
  Synthetic
agttgaagttgccatcagcaaacat

SEQ ID NO: 19 - DNA, 19 bp, Artificial Sequence
  Synthetic
ccgagagcggctgcttgtc

SEQ ID NO: 20 - DNA, 25 bp, Artificial Sequence
  Synthetic
gccacaaagatagtcactgtctgcc

SEQ ID NO: 21 - DNA, 26 bp, Artificial Sequence
  Synthetic
ggagaacaatgaaacctcagtagatt

SEQ ID NO: 22 - DNA, 27 bp, Artificial Sequence
  Synthetic
ctattgtccagatacattccagagtcc

SEQ ID NO: 23 - DNA, 24 bp, Artificial Sequence
  Synthetic
caccagagattaaaggaccatggc

SEQ ID NO: 24 - DNA, 21 bp, Artificial Sequence
  Synthetic
ggaagcctggagcaccacctc

SEQ ID NO: 25 - DNA, 20 bp, Artificial Sequence
  Synthetic
caaaggacgggtcctggttc

SEQ ID NO: 26 - DNA, 22 bp, Artificial Sequence
  Synthetic
ccttccggttaactcgagtaag

SEQ ID NO: 27 - DNA, 22 bp, Artificial Sequence
  Synthetic
cttactcgagttaaccggaagg

SEQ ID NO: 28 - DNA, 23 bp, Artificial Sequence
  Synthetic
gcatggacacaatacacggaatc